(19) United States
(12) Patent Application Publication
(10) Pub. No.: US 2025/0050021 A1
(43) Pub. Date: Feb. 13, 2025

(54) ADJUSTABLE GLUCOSE COST COMPONENT IN A COST FUNCTION FOR DETERMINING BASAL DELIVERY DOSES IN A MEDICAMENT DELIVERY SYSTEM

(71) Applicant: INSULET CORPORATION, Acton, MA (US)

(72) Inventors: Yibin ZHENG, Hartland, WI (US); Joon Bok LEE, Acton, MA (US); William WHITELEY, Knoxville, TN (US); Jason O’CONNOR, Acton, MA (US)

(21) Appl. No.: 18/797,048

(22) Filed: Aug. 7, 2024

Related U.S. Application Data
(60) Provisional application No. 63/518,453, filed on Aug. 9, 2023.

(51) Int. Cl. A61M 5/172; G16H 20/17

(52) U.S. Cl. CPC A61M 5/1723; G16H 20/17; A61M 2230/201

(57) ABSTRACT

Exemplary embodiments may configure the glucose cost component of a cost function used in determining basal insulin delivery doses to be limited by the maximum physiological rate of glucose level change over a time period so that the cost function more accurately reflects the physical limits of change due to insulin action, As a result, the glucose cost component of the cost function may more accurately reflect the response of a user to basal insulin deliveries, resulting in better insulin control for the user. Exemplary embodiments may modify the aggressiveness of a control approach based on a current target glucose level versus a nominal target glucose level for which the control approach was designed.

### CROSS-REFERENCE TO RELATED APPLICATIONS

This application claims priority to and the benefit of U.S. Provisional Application No. 63/518,453, filed Aug. 9, 2023, the entirety of which is incorporated herein by reference.

### BACKGROUND

Conventional automated insulin delivery (AID) systems may employ a cost function to determine the costs of candidate basal insulin doses. These conventional AID systems may then choose the lowest cost candidate basal insulin dose among the candidate doses for basal insulin delivery. The cost function may include a glucose cost component and an insulin cost component. The glucose cost component specifies the cost of expected deviations in glucose levels in a future time horizon for a user relative to a target glucose level if a candidate basal insulin dose is delivered. The insulin cost component specifies the cost of deviations in insulin delivery doses relative to a pre-set basal delivery dose over a time horizon if the candidate basal insulin delivery dose is delivered. Thus, the cost function penalizes deviations in glucose levels relative to target glucose levels and penalizes deviations in insulin doses relative to the pre-set basal delivery dose, which deviations are expected to result from delivery of the candidate basal insulin dose.

One difficulty with the glucose cost component of such conventional cost functions is that the glucose cost component penalizes deviations in the glucose level of the user regardless of the physiological limits regarding how much insulin action may decrease the glucose level of the user in a cycle of the AID system. The optimal basal insulin dose is calculated as a basal insulin dose that most rapidly brings the glucose level of the user to the target glucose level independent of the physiological limits regarding how much insulin action may decrease glucose level of the user in a cycle. Thus, where the glucose level of the user is elevated, the optimal dose chosen as having the lowest cost may not produce the desired result due to the physiological limits.

Another difficulty with conventional cost functions is that the conventional cost functions are designed to mitigate the risk of over-delivery of insulin due to erroneous glucose level data from glucose sensors and to balance the rate of driving the glucose level of the target with the risk of undershoot of the glucose level of the user that may result in possible hypoglycemia. As a result, when the user has a higher than average target glucose level, the cost function may not be sufficiently aggressive enough to bring the glucose level of the user to the elevated target quickly. Hence, the user may have an elevated glucose level for longer than is desirable.

### SUMMARY

In accordance with an inventive aspect, a medicament delivery device for delivering insulin to a user includes a non-transitory computer-readable storage medium storing computer programming instructions and a processor configured for executing the computer programming instructions. Executing the computer programming instructions may cause the processor to constrain a glucose cost component of a cost function for calculating a cost of a candidate insulin dose based on a physiological limit as to rate of change of glucose level of the user over a time period. Executing the computer programming instructions may further cause the processor to determine a next dose of insulin to be delivered to the user by the medicament delivery device based on the cost as calculated using the cost function and may cause the processor to cause delivery of the determined next dose from the medicament delivery device to the user.

The cost function may contain an insulin cost component. The determining of the next dose may entail choosing a dose with a lowest cost among candidate insulin doses as the next dose. The glucose cost component may be calculated by determining for each cycle of a future time horizon a weighted sum of a square of either a physiological limit as to rate of change of glucose level of the user for the cycle or of a difference between a predicted glucose level of the user for the cycle if the candidate insulin dose is delivered to the user and a target glucose level value for the cycle. The cycle may be a time period for which a single basal insulin dose is delivered. The glucose cost component may be calculated by determining for each cycle in a future time horizon a weighted sum of a square of a difference between a predicted glucose level of the user for the cycle if the candidate insulin dose is delivered to the user and a target glucose level for the cycle. The target glucose level for the cycle may be a larger of the target glucose for the selected one of the respective cycles and the predicted glucose level of the user for the cycle minus a physiological maximum possible decrease in glucose level of the user for a portion of the time horizon that ends with the cycle.

In accordance with another inventive aspect, a medicament delivery device for delivering insulin to a user may include a non-transitory computer-readable storage medium storing computer programming instructions and a processor that is configured for executing the computer programming instructions. Executing the computer programming instructions may cause the processor to adjust a weight of a glucose cost component of a cost function when a current target glucose level of a user deviates from a nominal target glucose level of the user, use the cost function with the adjusted weight to determine an insulin dose of the user, and deliver the determined insulin dose from the medicament delivery device to the user.

The adjusting of the weight may increase the weight where the current target glucose level of the user exceeds the nominal target glucose level of the user. The weight may be increased in a magnitude reflective of a magnitude of the difference between the current target glucose level of the user and the nominal target glucose level of the user. The weight may be a product of a nominal weight and a sum of one and a ratio of a square of a difference between the current target glucose level of the user and the nominal target glucose level of the user versus the nominal target glucose level of the user. The adjusting of the weight may increase the weight if the current target glucose level exceeds a threshold. The medicament delivery device may include a reservoir for storing the insulin.

In accordance with an additional inventive aspect, a medicament delivery device for delivering insulin to a user may include a non-transitory computer-readable storage medium storing computer programming instructions and a processor configured for executing the computer programming instructions. Executing the computer programming instructions may cause the processor to adjust a weight of a glucose cost component of a cost function, that is for determining costs of insulin dose candidates, based on a threshold glucose level value, use the cost function with adjusted weight to select a suitable insulin dose for the user among the candidate insulin doses, and deliver the selected insulin dose from the medicament delivery device to the user.

The adjusting of the weight of the glucose cost component may include multiplying a nominal weight for the glucose cost component by an adjustment factor. A magnitude of the adjustment factor may be based in part on a magnitude of a difference between the threshold glucose level value and a nominal target glucose level. A magnitude of the adjustment factor also may be based in part on a magnitude of a difference between the threshold glucose level value and a current target glucose level. The adjustment factor may be equal to a ratio of a square of a difference between the glucose level threshold and the nominal target glucose level divided by a square of the difference between the threshold glucose level value and the current target glucose level. The threshold glucose level value may be greater than a nominal target glucose level, and the adjusting of the weight of the glucose cost component may increase the weight of the glucose cost component.

### BRIEF DESCRIPTION OF THE DRAWINGS

FIG. 1 depicts a block diagram of an illustrative medicament delivery system that is suitable for delivering a medicament, such as insulin, to a user in accordance with the exemplary embodiments.

FIG. 2 depicts a flowchart of illustrative steps that may be performed in exemplary embodiments to select and deliver a basal insulin delivery dose using a cost function.

FIG. 3 depicts a flowchart of illustrative steps that may be performed in exemplary embodiments in limiting the glucose cost component.

FIG. 4 depicts a flowchart of illustrative steps that may be performed in exemplary embodiments to limit the glucose cost component based on a maximum.

FIG. 5 depicts a flowchart of illustrative steps that may be performed in exemplary embodiments to adjust the target glucose level to account for the maximum physiological change per cycle.

FIG. 6 depicts a flowchart of steps that may be performed in exemplary embodiments to determine a modified target glucose level value.

FIG. 7 depicts a flowchart of illustrative steps that may be performed in exemplary embodiments to increase aggressiveness of the control approach for the medicament delivery device.

FIG. 8 depicts a flowchart of illustrative steps that may be performed in exemplary embodiments to adjust the weight of the glucose cost component.

FIG. 9 depicts a flowchart of illustrative steps that may be performed in exemplary embodiments to determine the adjusted scaled weight.

### DETAILED DESCRIPTION

Exemplary embodiments may configure the glucose cost component of a cost function used in determining basal insulin delivery doses to be constrained by the maximum physiological rate of change in glucose level of the user over a time period so that the cost function more accurately reflects the physical limits of change. As a result, the glucose cost component of the cost function may more accurately reflect the response of a user to basal insulin deliveries, resulting in better insulin control for the user. To achieve this constraining, exemplary embodiments may limit the deviation values that are calculated as part of the glucose cost component between a predicted glucose level at a cycle and a target glucose level for the cycle over cycles of a future time horizon to the physiological limit of change in glucose level that is possible in a cycle. Alternatively, the deviations calculated in the glucose cost component of the cost function for the future time horizon may be determined as the difference in each cycle of the future time horizon between the predicted glucose level for the cycle given delivery of the candidate basal insulin dose and a modified target glucose level. The modified target glucose level may be the larger of a nominal target glucose level and the predicted glucose level of the user for the cycle minus the maximum amount that the glucose level of the user could change for that cycle in the time horizon (e.g., the product of the physiological limit of change in glucose level per cycle and the cycle number in the time horizon cycle sequence).

Exemplary embodiments may modify the aggressiveness of a control approach of the medicament delivery device based on a current target glucose level versus a nominal target glucose level for which the control approach was designed. Exemplary embodiments may increase the aggressiveness of glucose level control of a medicament delivery system by adjusting a glucose cost component weight to be larger when a current target glucose level of a user exceeds the nominal target glucose level. Alternatively, the value of the weight may be scaled relative to an adjustment factor that may be a ratio of a square of a difference between the threshold glucose level and the nominal target glucose level versus a square of a difference between the threshold glucose level and the current target glucose level. As a result, the aggressiveness is better matched to the current target glucose level.

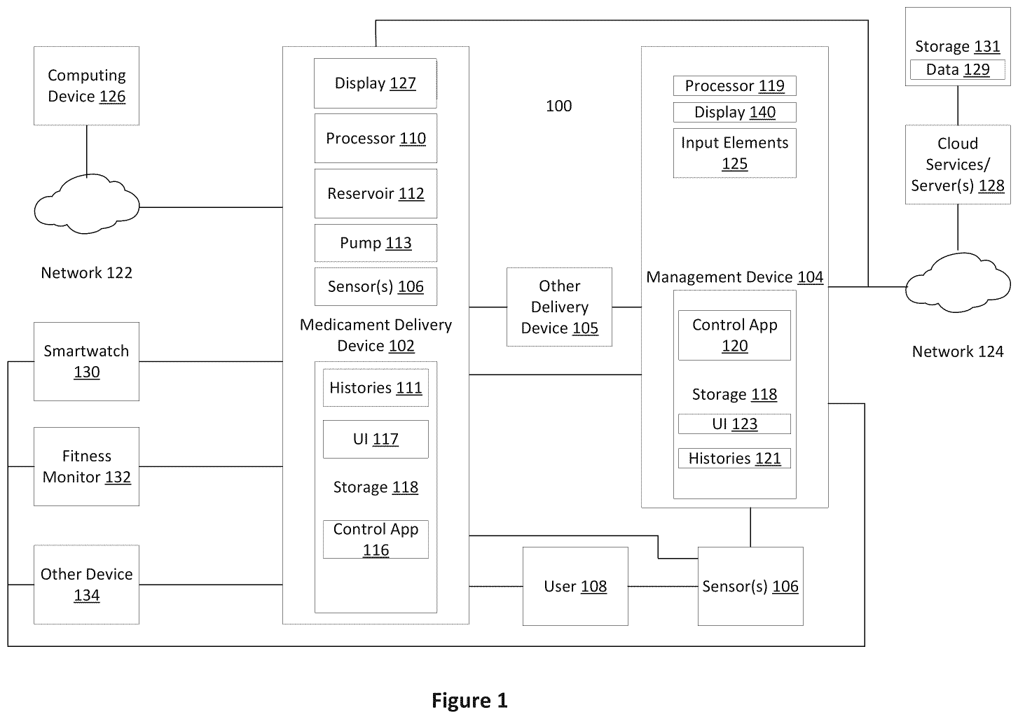

FIG. 1 depicts a block diagram of an illustrative medicament delivery system 100 that is suitable for delivering a medicament, such as insulin, to a user 108 in accordance with the exemplary embodiments. The medicament delivery system 100 may include a medicament delivery device 102. The medicament delivery device 102 may be a wearable device that is worn on the body of the user 108 or carried by the user. The medicament delivery device 102 may be directly coupled to the user 108 (e.g., directly attached to a body part and/or skin of the user 108 via an adhesive or the like) with no tubes and an infusion location directly under the medicament delivery device 102, or carried by the user 108 (e.g., on a belt or in a pocket) with the medicament delivery device 102 connected to an infusion site where the medicament is injected using a needle and/or cannula. A surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of the medicament to the user 108 per a control approach like that described herein. The control application may use a glucose prediction model as described below for predicting future glucose levels of the user 108. The storage 114 may hold histories 111 for a user, such as a history of basal deliveries, a history of bolus deliveries, and/or other histories, such as a meal event history, exercise event history, glucose level history, other analyte level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include a tray or cradle and/or one or more housings for housing its various components including a pump 113, a power source (not shown), and a reservoir 112 for storing medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site. The medicament delivery device 102 may have operational cycles, such as every 5 minutes, in which basal doses of medicament are calculated and delivered as needed. These steps are repeated for each cycle.

There may be one or more communications links with one or more devices physically separated from the medicament delivery device 102 including, for example, a management device 104 of the user 108 and/or a caregiver of the user 108, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN), a cellular network, a Wi-Fi network, a near field communication network, or a combination thereof. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions, physical attributes, or analyte levels of the user 108. The sensor(s) 106 may be physically separate from the medicament delivery device 102 or may be an integrated component thereof. The sensor(s) 106 may include, for example, glucose monitors, such as continuous glucose monitors (CGM's) and/or non-invasive glucose monitors. The sensor(s) 106 may include ketone sensors, other analyte sensors, heart rate monitors, breathing rate monitors, motion sensors, temperature sensors, perspiration sensors, blood pressure sensors, alcohol sensors, or the like. Some sensors 106 may also detect characteristics of components of the medicament delivery device 102. For instance, the sensors 106 in the medicament delivery device may include voltage sensors, current sensors, temperature sensors and the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. Storage 118 may also be operable to store historical information such as medicament delivery information, analyte level information, user input information, output information, or other historical information. The control application 120 may be responsible for controlling the medicament delivery device 102, such as by controlling the automated medicament delivery (AMD) (or, for example, automated insulin delivery (AID)) of medicament to the user 108. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when it is a touchscreen. The management device 104 may further include input elements 125, such as a keyboard, button, knobs, or the like, for receiving input for the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user 108, such as to request a change or pause in dosage, or to request, initiate, or confirm delivery of a bolus of medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data. Another delivery device 105, such as a medicament delivery pen (such as an insulin pen), may be accounted for (e.g., in determining insulin on board (IOB)) or may be provided for also delivering medicament to the user 108.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134.

In the closed loop mode, the control application 116, 120 determines the medicament delivery amount for the user 108 on an ongoing basis based on a feedback loop. For a medicament delivery device that uses insulin, for example, the aim of the closed loop mode is to have the user's glucose level at a target glucose level or within a target glucose range.

In some embodiments, the medicament delivery device 102 need not deliver one medicament alone. Instead, the medicament delivery device 102 may deliver one medicament, such as insulin, for lowering glucose levels of the user 108 and also deliver another medicament, such as glucagon, for raising glucose levels of the user 108. The medicament delivery device 102 may deliver a glucagon-like peptide (GLP)-1 receptor agonist medicament for lowering glucose or slowing gastric emptying, thereby delaying spikes in glucose after a meal. The medicament delivery device 102 may deliver a gastric inhibitory polypeptide (GIP) or a dual GIP-GLP receptor agonist. In other embodiments, the medicament delivery device 102 may deliver pramlintide, or other medicaments that may substitute for insulin. In other embodiments, the medicament delivery device 102 may deliver concentrated insulin. In some embodiments, the medicament or medicament delivered by the medicament delivery device may be a coformulation of two or more of those medicaments identified above. In a preferred embodiment, the medicament delivery device delivers insulin; accordingly, reference will be made throughout this application to insulin and an insulin delivery device, but one of ordinary skill in the art would understand that medicaments other than insulin can be delivered in lieu of or in addition to insulin.

Insulin deliveries to the user 108 may be bolus insulin deliveries or basal insulin deliveries. Bolus insulin deliveries tend to be to offset the expected rise in glucose level of the user 108 from ingesting a meal or for correcting a persistently elevated glucose level (i.e., one that is persistently higher than a target glucose level). Boluses tend to be one time deliveries for offsetting a meal or for correcting a glucose level and tend to be larger than bolus insulin deliveries. Insulin boluses may be delivered manually by the user 108, such as via a syringe, or may, in some exemplary embodiments, be delivered by the medicament delivery device 102. Basal insulin doses tend to be smaller than insulin bolus doses and are delivered periodically, such as once each operational cycle of the control approach of the medicament delivery device 102 (e.g., every 5 minutes). The aim of the basal insulin deliveries is to keep the user's glucose level within a target range that is desirable using small ongoing insulin doses.

The control approach of the exemplary embodiments that is performed by the control application 116 or 120 may select a suitable insulin dose among candidate basal insulin delivery doses based on a cost function. A typical conventional cost function is:

$$J ( k ) = Q {\sum }_{i = k + 1}^{P} {( G ( i ) - SP ( i ) )}^{2} + R {\sum }_{i = k + 1}^{C} {( I ( i ) - b ( i ) )}^{2} \qquad ( Equation 1 )$$

where J(k) is the cost of a specified insulin dose for cycle k, QΣᵢ₌ₖ₊₁^{P}(G(i)−SP(i))² is the glucose cost component, and RΣᵢ₌ₖ₊₁^{C}(I(i)−b(i))² is the insulin cost component. Q is a weight coefficient for the glucose cost component. The glucose cost component represents the weighted sum of the deviations squared in the glucose level of the user 108 (G(i)) over a future time horizon (cycles k+1 to P) relative to a target glucose level SP(i) if the specified basal insulin dose is delivered, R is a weight coefficient for the insulin cost component. The insulin cost component represents the costs of the squares of the deviations in insulin delivery amounts (I(i)) delivered over a time period (cycles k+1 to C) in the future relative to an ideal basal insulin dose (b(i)).

One problem with using such a conventional cost function is that the glucose cost component assumes that there is no limit as to how much the glucose level of the user 108 may decrease in a cycle responsive to insulin action. In reality, glucose levels may decrease only a maximum of 20 mg/dL per 5-minute cycle due to insulin action and physiological constraints. As a result, the conventional cost function does not accurately reflect the limits of insulin action per a cycle. The exemplary embodiments may modify the cost function used by control application 116 or 120 to account for this physiological limit. The exemplary embodiments may thus more accurately reflect reality, and the modified cost function of the exemplary embodiments may result in better glucose level control.

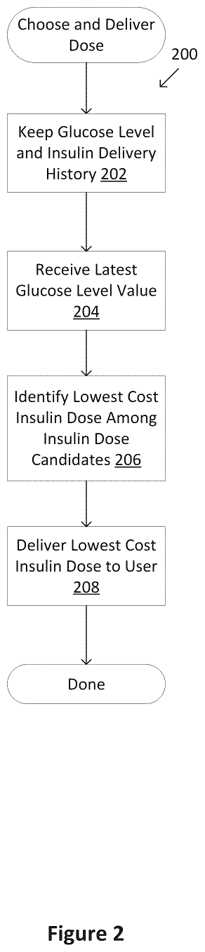

FIG. 2 depicts a flowchart 200 of illustrative steps that may be performed in exemplary embodiments to select and deliver a basal insulin delivery dose using a modified cost function. At 202, a glucose level history and an insulin delivery history of the user 108 are kept by one or both of the medicament delivery device 102 and the management device 104. In some instances, at least portions of the histories may be stored on external storage devices, such as storage 131. At 204, the medicament delivery device 102 may receive a latest glucose level value of the user 108 from a sensor 106, such as a glucose monitor, like a CGM. At 206, the control application 116 or 120 determines a lowest cost insulin dose among the candidate insulin doses for the current cycle. The lowest cost may be determined using a cost function like that described below, and optimization strategies may be applied to the cost function to identify the lowest cost dose among the insulin dose candidates. The pool of insulin dose candidates may be determined based upon the constraints on basal insulin delivery doses imposed by the control application 116 or 120. At 208, the control application 116 or 120 then delivers the lowest cost insulin dose to the user 108 as the basal insulin delivery dose for the cycle unless the dose is determined to be 0, such as when the control application 116 or 120 determines that insulin delivery needs to be suspended.

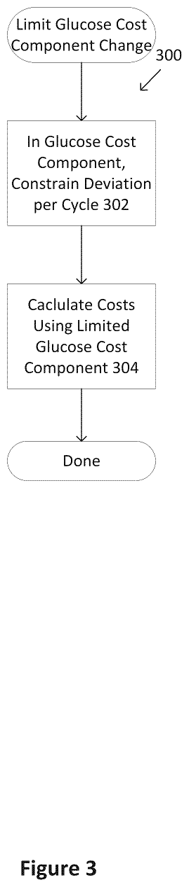

The exemplary embodiments may limit the magnitude of deviations calculated in the glucose cost component of the modified cost function between consecutive operational cycles of a predicted time horizon so that the glucose cost component reflects the physiological limits of the deviation in glucose level of the user 108 that may occur in a single cycle. FIG. 3 depicts a flowchart 300 of illustrative steps that may be performed in exemplary embodiments in constraining the deviations of the glucose cost component. At 302, the deviation per cycle is constrained. The constraining may reflect the physiological limit to change per cycle of 20 mg/dL per 5-minute cycle. At 304, the costs are calculated using the constrained glucose cost component.

There may be different ways of constraining the glucose cost component. One way is to limit the deviation per cycle that is aggregated in calculating the glucose cost component to a maximum, such as the physiological limit of 20 mg/dL per 5-minute cycle. The glucose cost component may be calculated to look at what the predicted glucose levels will be for each cycle in a future time horizon given the current glucose level of the user 108 and the past glucose levels. Predicted glucose levels of the user 108 may be predicted for the cycles of the future glucose cycles of the time horizon using a glucose prediction model. A suitable model is:

$${G}_{Δ} ( k ) = {b}_{1} {G}_{Δ} ( k - 1 ) + {b}_{2} {G}_{Δ} ( k - 2 ) + {b}_{3} {G}_{Δ} ( k - 3 ) - {KI}_{Δ} ( k - 3 ) \qquad ( Equation 2 )$$

where G_{Δ}(k) is a delta between a predicted glucose level at cycle k and a target glucose level value, b₁, b₂, and b₃ are weight coefficients for the past glucose values, I_{Δ}(k−3) is a delta between the insulin dose delivered at cycle k−3, and K is a weight coefficient.

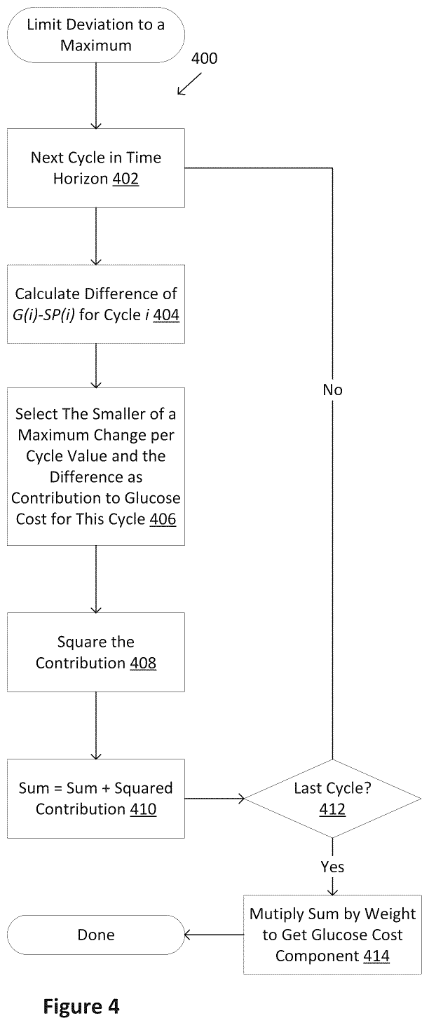

FIG. 4 depicts a flowchart 400 of illustrative steps that may be performed in exemplary embodiments to limit the glucose cost component in such a fashion. These steps iterate through each cycle in the time horizon and determine the difference between the predicted glucose level of the user 108 for the cycle and the target glucose level for the cycle. This difference may be limited to the physiological limit of 20 mg/dL per 5-minute cycle (or 4 mg/dL in a 1-minute cycle) in some exemplary embodiments. At 402, a value of a cycle index is incremented. The cycle index identifies which cycle in a future time horizon is being processed in determining the glucose cost component. The index i may initialized to have a value of k+1. At 404, a difference between the predicted glucose level of the user 108 G(i) and the target glucose level for the cycle SP(i) is calculated. At 406, the smaller of the maximum change in glucose level of the user 108 per cycle (e.g., 20 mg/dL) and the difference which was determined in 404 is selected as the contribution to the glucose cost component for this cycle. In other words, the function min(20,G(i)−SP(i)) is applied and the minimum value is selected per the function. At 408, this contribution is squared. At 410, a running sum of the squared contributions of each cycle in the time horizon is incremented to include the squared contribution for the cycle. At 412, a check is made whether the cycle is the last cycle. If not, the process repeats beginning at 402. If the last cycle is reached, the glucose cost component is determined by multiplying the sum by the weight coefficient Q. Hence, the glucose cost component may be summarized as:

$$glucose cost component = Q {\sum }_{i = k + 1}^{P} min {( 20 , G ( i ) - SP ( i ) )}^{2} . \qquad ( Equation 3 )$$

Moreover, the cost J′(k) may be calculated using the following the cost function:

$${J}^{′} ( k ) = Q {\sum }_{i = k + 1}^{P} min {( 20 , G ( i ) - SP ( i ) )}^{2} + R {\sum }_{i = k + 1}^{C} {( I ( i ) - b ( i ) )}^{2} . \qquad ( Equation 4 )$$

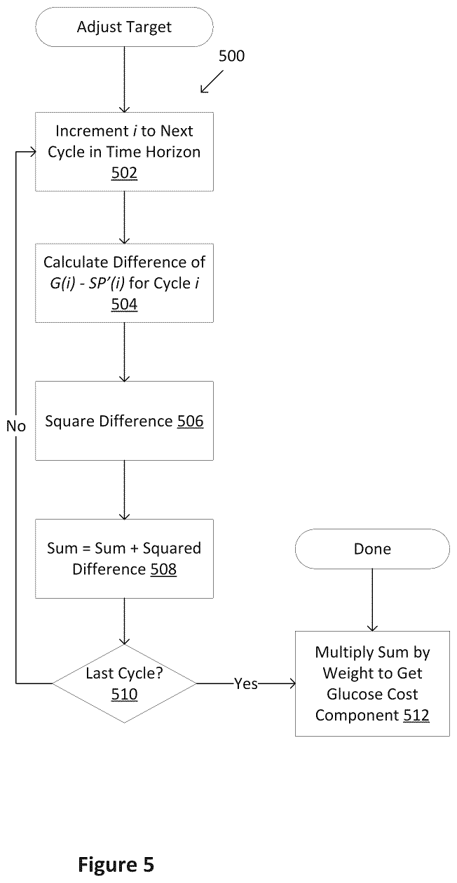

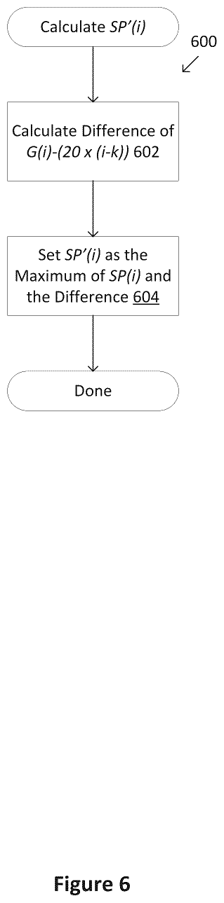

Another approach to constraining the deviation per cycle of the glucose cost component to the rate of maximum physiological change is to adjust the target glucose level of the user 108. FIG. 5 depicts a flowchart 500 of illustrative steps that may be performed in exemplary embodiments to adjust the target glucose level to account for the maximum physiological change per cycle. At 502, the cycle index is incremented to refer to the next operational cycle in the time horizon of interest. The index i may be initialized at k+1 (i.e., the first cycle in the time horizon) before 502. At 504, the difference between the expected glucose level at cycle k, i.e., G(i), as predicted using the glucose prediction model (such as in Equation 2) and the adjusted target glucose level SP′(i) is determined. The steps for determining SP′ (i) are discussed below relative to FIG. 6. SP′(i) is adjusted so that the difference as calculated at 504 does not exceed the maximum change that can occur by the end of the cycle in question. At 506, the difference is squared. At 508, the aggregated sum is incremented by the squared difference. At 510, a check is made whether the cycle index k refers to the last cycle in the time horizon over which the glucose cost component is determined. If not, the process repeats at 502. If so, the sum is multiplied by the weight for the glucose cost component Q to yield the glucose cost component at 512. Hence, the glucose cost component may be expressed as QΣᵢ₌ₖ₊₁^{P}(G(i)−SP′(i))². The resulting cost function may expressed as:

$${J}^{′} ( k ) = Q {\sum }_{i = k + 1}^{P} {( G ( i ) - {SP}^{′} ( i ) )}^{2} + R {\sum }_{i = k + 1}^{C} {( I ( i ) - b ( i ) )}^{2} . \qquad ( Equation 5 )$$

FIG. 6 depicts a flowchart 600 of the steps that may be performed in exemplary embodiments to determine SP′(i). At 602, a difference between the predicted glucose level for cycle i, (G(i)), and 20 times the difference of the cycle indices i and k. Thus for cycle k+1, the difference is calculated between the glucose level at cycle k+1 and 20×1. For cycle, k+2, the difference is calculated between the glucose level at cycle k+2 and 20×2 and so forth for the remaining successive cycles of the time horizon. At 604. SP′(i) is set as the maximum of SP(i) and the difference calculated at 602.

Other maximum values may be used rather than the physiological limit of change in glucose level per cycle in some exemplary embodiments.

As was mentioned above, conventional cost functions are biased toward avoiding hypoglycemia. The conventional cost functions may not work well where the current target glucose level of the user 108 is higher than the target for which the control approach is configured, referred to as the nominal target glucose level. The exemplary embodiments may address this issue by increasing the aggressiveness of the cost function when the current target glucose level of the user 108 is above the nominal target glucose level. The term aggressiveness in this context is intended to mean that the control approach of the control application 116 or 120 responds more aggressively (i.e., with greater action, such as delivering more insulin sooner or reducing insulin delivery more substantially) to deviations relative to the target glucose level of the user 108. In the exemplary embodiments, the aggressiveness may be increased by modifying the glucose cost component weight Q.

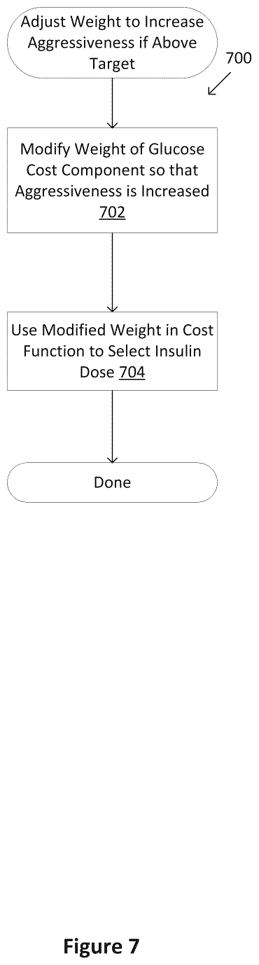

Exemplary embodiments may modify the aggressiveness of a control approach based on a current target glucose level versus a nominal target glucose level for which the control approach was designed. FIG. 7 depicts a flowchart 700 of illustrative steps that may be performed in exemplary embodiments to increase aggressiveness. At 702, the weight of the glucose cost component is modified to increase aggressiveness, as is explained below. At 704, the modified weight of the glucose cost component is used in the cost function to select basal insulin delivery doses by the medicament delivery device 102 to the user 108.

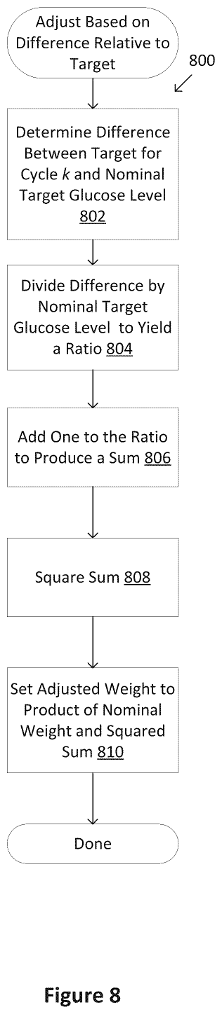

FIG. 8 depicts a flowchart of illustrative steps that may be performed in exemplary embodiments to adjust the weight of the glucose cost component. These illustrative steps may be performed where the weight of the glucose cost component for cycle k, Q_{f}(k), is calculated as

$${Q}_{f} ( k ) = {{Q}_{nom} ( 1 + \frac{SP ( k ) - {SP}_{nom}}{{SP}_{nom}} )}^{2} \qquad ( Equation 6 )$$

where Qₙₒₘ is the nominal weight for the glucose component and SPₙₒₘ is the nominal target glucose level.

At 802, the difference between a target glucose level SP (k) of the user 108 for the current cycle k and a nominal target glucose level SPₙₒₘ is determined. At 804, this difference is divided by SPₙₒₘ to yield a ratio that reflects how big the difference is relative to the nominal target glucose level. At 806, one is added to the ratio to produce a sum

$$( i . e . , 1 + \frac{SP ( k ) - {SP}_{nom}}{{SP}_{nom}} ) .$$

At 808, this sum is squared. At 810, the adjusted weight is set as the product of the nominal weight Qₙₒₘ of the glucose cost component and the squared sum, which is referred to herein as the adjustment factor.

The weight may also be adjusted to be scaled relative to a glucose level threshold. As a starting point, Q_{f}(Gₜₕ−SP(k))²=Qₙₒₘ(Gₜₕ−SPₙₒₘ)² (Equation 7), which may be solved for the adjusted weight Q_{f} as:

$${Q}_{f} = {Q}_{nom} \frac{{( {G}_{th} - {SP}_{nom} )}^{2}}{{( {G}_{th} - SP ( k ) )}^{2}} \qquad ( Equation 8 )$$

where Gₜₕ is the glucose level threshold.

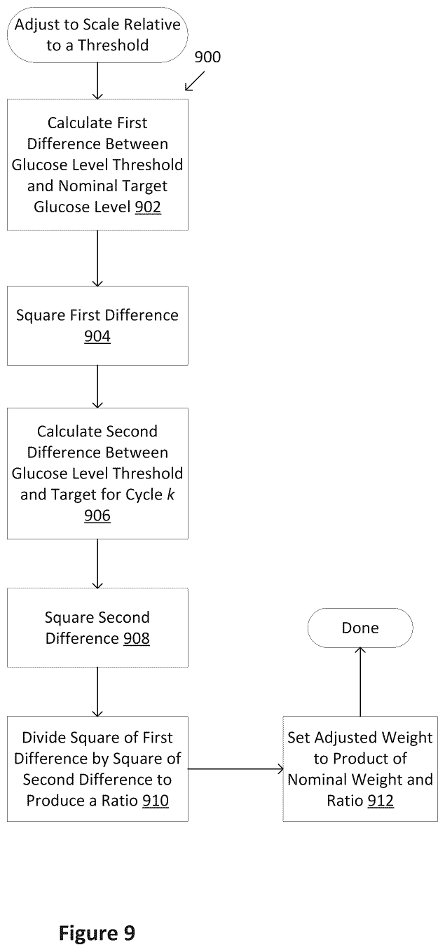

FIG. 9 depicts a flowchart 900 of illustrative steps that may be performed in exemplary embodiments to determine the adjusted weight using Equation 8. At 902, a first difference between the glucose level threshold Gₜₕ and the nominal target glucose level SPₙₒₘ is calculated. At 904, this first difference is squared. At 906, a second difference between the glucose level threshold Gₜₕ and the current target glucose level SP(k) is calculated. At 908 the second difference is squared. The square of the first difference is divided by the square of the second difference to produce a ratio (i.e., the adjustment factor). The ratio reflects how much the glucose level threshold differs from the nominal target glucose level relative to how the glucose level threshold varies from the current target glucose level. So, the weight increases if the current target glucose level is larger than the nominal target glucose level and decreases if the current target glucose level is smaller than the nominal target glucose level in instances where the glucose level threshold is greater than the target glucose levels SP (k) and SPₙₒₘ. At 912, the adjusted weight Q_{f} is set equal to the product of the nominal weight Qₙₒₘ and the ratio. The nominal weight Qₙₒₘ is the weight for which the control approach was designed.

The adjustment factor need not be a ratio of squares but may be a ratio of absolutes values in some exemplary embodiments.

While exemplary embodiments have been described herein, various changes in form and detail may be made without departing from the intended scope of the appended claims.

1. A medicament delivery device for delivering insulin to a user, comprising:
a non-transitory computer-readable storage medium storing computer programming instructions;
a processor configured for executing the computer programming instructions to cause the processor to:
constrain a glucose cost component of a cost function for calculating a cost of a candidate insulin dose based on a physiological limit as to rate of change of glucose level of the user over a time period;
determine a next dose of insulin to be delivered to the user by the medicament delivery device based on cost as calculated using the cost function; and
cause delivery of the determined next dose from the medicament delivery device to the user.

2. The medicament delivery device of claim 1, wherein the cost function contains an insulin cost component.

3. The medicament delivery device of claim 1, wherein the determining of the next dose comprises choosing a dose with a lowest cost among candidate insulin doses as the next dose.

4. The medicament delivery device of claim 1, wherein the glucose cost component is calculated by determining for each cycle of a future time horizon a weighted sum of a square of either a physiological limit as to rate of change of glucose level of the user for the respective cycle or of a difference between a predicted glucose level of the user for the cycle if the candidate insulin dose is delivered to the user and a target glucose level value for the cycle.

5. The medicament delivery device of claim 1, wherein the cycle is a time period for which a single basal insulin dose is delivered.

6. The medicament delivery device of claim 1, wherein the glucose cost component is calculated by determining for each cycle in a future time horizon a weighted sum of a square of a difference between a predicted glucose level of the user for the cycle if the candidate insulin dose is delivered to the user and a target glucose level for the cycle.

7. The medicament delivery device of claim 6, wherein the target glucose level for the cycle is a larger of the target glucose level for the selected one of the respective cycles and the predicted glucose level of the user for the cycle minus a physiological maximum possible decrease in glucose level of the user for a portion of the time horizon that ends with the cycle.

8. A medicament delivery device for delivering insulin to a user, comprising:
a non-transitory computer-readable storage medium storing computer programming instructions;
a processor configured for executing the computer programming instructions to cause the processor to:
adjust a weight of a glucose cost component of a cost function when a current target glucose level of a user deviates from a nominal target glucose level of the user;
use the cost function with the adjusted weight to determine an insulin dose for the user; and
deliver the determined insulin dose from the medicament delivery device to the user.

9. The medicament delivery device of claim 8, wherein the adjusting of the weight increases the weight where the current target glucose level of the user exceeds the nominal target glucose level of the user.

10. The medicament delivery device of claim 9, wherein the weight is increased in a magnitude reflective of a magnitude of the difference between the current target glucose level of the user and the nominal target glucose level of the user.

11. The medicament delivery device of claim 10, wherein the weight is a product of a nominal weight and a sum of one and a ratio of a square of a difference between the current target glucose level of the user and the nominal of the user versus the nominal target glucose level of the user.

12. The medicament delivery device of claim 8, wherein the adjusting of the weight increases the weight if the current target glucose level of the user exceeds a threshold.

13. The medicament delivery device of claim 8, further comprising a reservoir for storing the insulin.

14. A medicament delivery device for delivering insulin to a user, comprising:
a non-transitory computer-readable storage medium storing computer programming instructions;
a processor configured for executing the computer programming instructions to cause the processor to:
adjust a weight of a glucose cost component of a cost function, that is for determining costs of insulin dose candidates, based on a threshold glucose level value;
use the cost function with adjusted weight to select a suitable insulin dose for the user among the candidate insulin doses; and
deliver the selected insulin dose from the medicament delivery device to the user.

15. The medicament delivery device of claim 14, wherein the adjusting of the weight of the glucose cost component comprises multiplying a nominal weight for the glucose cost component by an adjustment factor.

16. The medicament delivery device of claim 15, wherein a magnitude of the adjustment factor is based in part on a magnitude of a difference between the threshold glucose level value and a nominal target glucose level.

17. The medicament delivery device of claim 16, wherein a magnitude of the adjustment factor also is based in part on a magnitude of a difference between the threshold glucose level value and a current target glucose level.

18. The medicament delivery device of claim 17, wherein the adjustment factor is equal to a ratio of a square of a difference between the glucose level threshold and the nominal target glucose level divided by a square of the difference between the threshold glucose level value and the current target glucose level.

19. The medicament delivery device of claim 14, wherein the threshold glucose level value is greater than a nominal target glucose level.

20. The medicament delivery device of claim 19, wherein the adjusting of the weight of the glucose cost component increases the weight of the glucose cost component.
